(19) United States
(12) Patent Application Publication
(10) Pub. No.: US 2009/0281127 A1
(43) Pub. Date: Nov. 12, 2009

(54) Organic Compounds

(76) Inventors: Robin Alec Fairhurst, West Sussex (GB); Roger John Taylor, West Sussex (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/297,007

(22) PCT Filed: Apr. 19, 2007
(86) PCT No.: PCT/EP07/03432

§ 371 Date: Oct. 13, 2008

(30) Foreign Application Priority Data
Apr. 21, 2006 (GB) 0607945.3

(51) Int. Cl. A61K 31/52; C07D 473/34

(52) U.S. Cl. 514/263.4; 544/277

(57) ABSTRACT

A compound of formula (I) and their preparation and use as pharmaceuticals

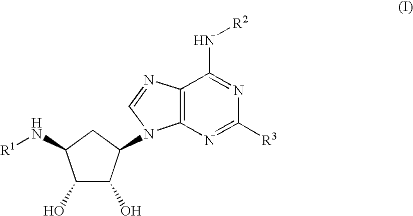

wherein R¹, R² and R³ are as defined herein.

This invention relates to organic compounds, their preparation and use as pharmaceuticals.

An aspect of the invention provides compounds of formula (I) or stereoisomers or pharmaceutically acceptable salts thereof,

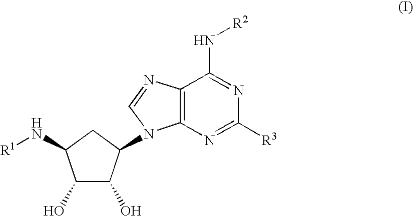

wherein R¹, R² and R³ are defined below

| R¹ | R² | R³ |
|---|---|---|
| 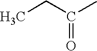 | 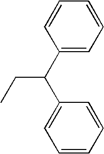 | 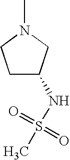 |
| 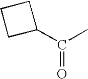 | 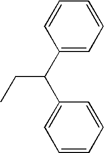 | 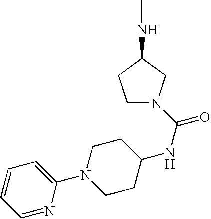 |
| 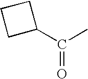 | 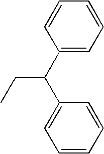 | 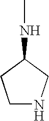 |
| 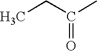 | 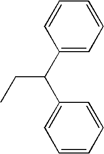 | 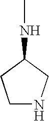 |
| 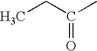 | 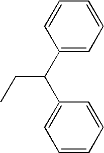 | 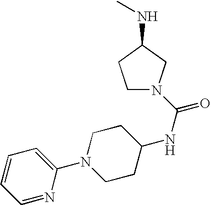 |
| 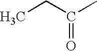 | 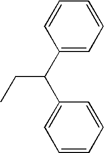 | 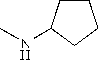 |
| 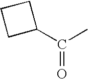 | 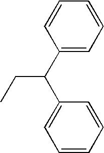 | 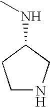 |
| 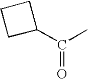 | 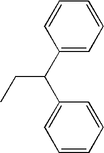 | 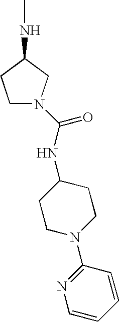 |
| 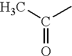 | 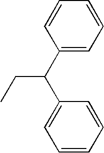 | 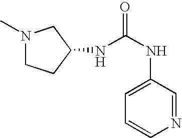 |
| 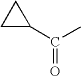 | 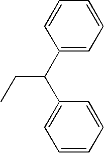 | 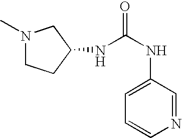 |
| 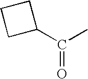 | 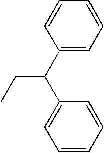 | 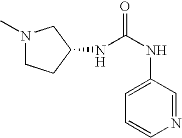 |
| 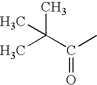 | 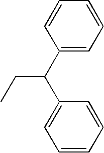 | 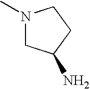 |
| 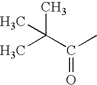 | 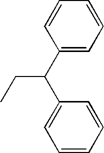 | 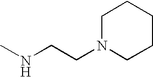 |
| 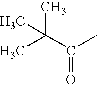 | 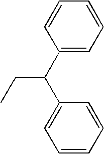 | 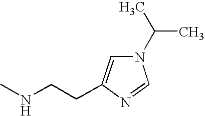 |
| 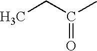 | 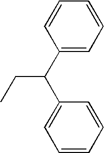 | 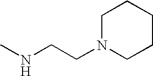 |

### Salts and Isomers

The compounds represented by formula (I) are capable of forming acid addition salts, particularly pharmaceutically acceptable acid addition salts. Pharmaceutically acceptable acid addition salts of the compound of formula I include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoroacetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid, para-biphenyl benzoic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, cinnamic acids such as 3-(2-naphthalenyl)propenoic acid, para-methoxy cinnamic acid or para-methyl cinnamic acid, and sulfonic acids such as methanesulfonic acid or benzenesulfonic acid. These salts may be prepared from compounds of formula I by known salt-forming procedures.

Stereoisomers are those compounds where there is an asymmetric carbon atom. The compounds exist in individual optically active isomeric forms or as mixtures thereof, e.g., as diastereomeric mixtures. The present invention embraces both individual optically active R and S isomers, as well as mixtures thereof. Individual isomers can be separated by methods well known to those skilled in the art, e.g. chiral high performance liquid chromatography (HPLC).

Tautomers are one of two or more structural isomers that exist in equilibrium and are readily converted from one isomeric form to another.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

### Synthesis

Another embodiment of the present invention, provides a process for the preparation of compounds of formula (I), in free or pharmaceutically acceptable salt form, which comprises the steps of:

(i) reacting a compound of formula (II)

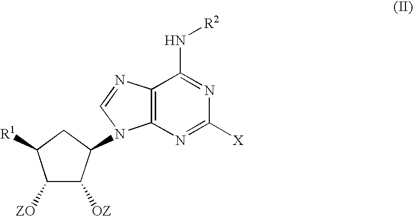

wherein
R¹, and R² are as defined in claim 1;
Z is H or a protecting group; and
X is a leaving group,
   with a compound of formula (III)

H—R³ (III),

wherein
R³ is as defined in claim 1; and
removing any protecting groups and recovering the resultant compound of formula (I), in free or pharmaceutically acceptable salt form.

The compound of formula (III) may be prepared by reacting a compound of formula (IV)

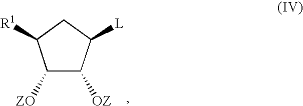

wherein
R¹ and Z are as defined in claim 1; and
L represents a leaving group or a protected derivative thereof with a 2,6-dihalopurine, e.g., 2,6-dichloropurine,
   to provide a compound of formula (V)

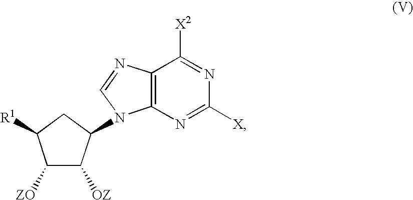

wherein
R¹ and Z are defined in claim 1; and
X and X² are halogen.

Compound of formula (V) can be reacted with R²NH₂ under conventional conditions to provide compound of formula (II).

The compounds of formula (I) can be prepared, e.g., using the reactions and techniques described below and in the Examples. The compounds of formula (I) can be prepared by the processes described in patent application PCT/EP2005/011344. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is Protective Groups In Organic Synthesis, Greene and Wuts, Eds., Wiley and Sons (1999).

### Pharmaceutcial Use

Compounds of formula I and their pharmaceutically acceptable salts are useful as pharmaceuticals. In particular, they activate the adenosine A2a receptor activation, i.e. they act as A2a receptor agonists. Their properties as A_{2A} agonists may be demonstrated using the method described by L. J. Murphree et al in Molecular Pharmacology 61, 455-462 (2002).

Compounds of the Examples hereinbelow have K; values below 1.0 μM in the above method. For example, the compounds of Examples 6, 10 and 15 have Kᵢ values of 0.004, 0.005, and 0.009 μM respectively.

Having regard to their activation of the adenosine A2a receptor, compounds of formula (I) in free or pharmaceutically acceptable salt form, hereinafter alternately referred to as “agents of the invention”, are useful in the treatment of conditions which are mediated by response to the activation of the adenosine A2a receptor, particularly inflammatory or allergic conditions. Treatment in accordance with the invention may be symptomatic or prophylactic.

Accordingly, agents of the invention are useful in the treatment of inflammatory or obstructive airways diseases, resulting, for example, in reduction of tissue damage, airways inflammation, bronchial hyperreactivity, remodelling or disease progression.

Inflammatory or obstructive airways diseases and conditions to which the present invention is applicable include acute lung injury (ALI), adultlacute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include bronchiectasis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Other inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. cortico-steroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant from any previously administered symptomatic asthma therapy.

Having regard to their anti-inflammatory activity, in particular in relation to inhibition of eosinophil activation, agents of the invention are also useful in the treatment of eosinophil related disorders, e.g. eosinophilia, in particular eosinophil related disorders of the airways (e.g. involving morbid eosinophilic infiltration of pulmonary tissues) including hyper-eosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Agents of the invention are also useful in the treatment of inflammatory or allergic conditions of the skin, for example psoriasis, contact dermatitis, atopic dermatitis, alopecia greata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

Agents of the invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunct-ivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy).

Further, agents of the invention may also be used for the treatment of cystic fibrosis, pulmonary hypertension, pulmonary fibrosis, inflammatory bowel syndrome, wound healing, diabetic nephropathy as described in WO 05/107463, reduction of inflammation in transplanted tissue as described in US 2005/182018, inflammatory diseases caused by pathogenic organisms as described in WO 03/086408, and cardiovascular conditions as described in WO 03/029264.

Also, the agents of the invention may be used to assess the severity of coronary artery stenosis as described in WO 00/078774 and useful in conjunction with radioactive imaging agents to image coronary activity and useful in adjunctive therapy with angioplasty as described in WO 00/78779.

Agents of the invention are also useful in combination with a protease inhibitor for prevention of organ ischaemia and reperfusion injury as described in WO 05/003150, and in combination with an integrin antagonist for treating platelet aggregation as described in WO 03/090733.

Agents of the invention are also useful in promoting wound healing in bronchial epithelial cells as described in AJP-Lung 290: 849-855.

Other diseases or conditions which may be treated with agents of the invention include diabetes, e.g. diabetes mellitus type I (juvenile diabetes) and diabetes mellitus type II, diarrheal diseases, ischemia/reperfusion injuries, retinopathy, such as diabetic retinopathy or hyperbaric oxygen-induced retinopathy, conditions characterised by elevated intraocular pressure or secretion of ocular aqueous humor, such as glaucoma, ischemic tissue/organ damage from reperfusion, bedsores, as agents for promoting sleep, as agents for treating demyelinating diseases, eg multiple sclerosis and as neuroprotective agents for eg, cerebral haemorrhagic injury and spinal cord ischaemi-reperfusion injury.

The effectiveness of an agent of the invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; Cernadas et al (1999) Am. J. Respir. Cell Mol. Biol. 20:1-8; and Fozard et al (2002) European Journal of Pharmacological438, 183-188.

The agents of the invention are also useful as co-therapeutic agents for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance.

Accordingly the invention includes a combination of an agent of the invention as hereinbefore described with an anti-inflammatory, bronchodilatory, antihistamine or anti-tussive drug substance, said agent of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTB4 antagonists such as BIlL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700; LTD4 antagonists such include montelukast, pranlukast, zafirlukast, accolate, SR2640, Wy-48,252, ICI 198615, MK-571, LY-171883, Ro 24-5913 and L-648051; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(TM) CC-10004 (Celgene), VM554/UM565 (Vemalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A_{2B} receptor antagonists such as those described in WO 02/42298; and beta-2 adrenoceptor agonists such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula I of WO 0075114, which document is incorporated herein by reference, preferably compounds of the Examples thereof, especially a compound of formula

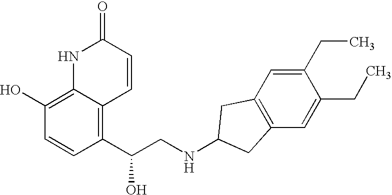

and pharmaceutically acceptable salts thereof, as well as compounds (in free or salt or solvate form) of formula I of WO 04/16601, and also compounds of EP 1440966, JP 05025045, WO 93/18007, WO 99/64035, US 2002/0055651, US 2005/0133417, US 2005/5159448, WO 01/42193, WO 01/83462, WO 02/66422, WO 02/70490, WO 02/76933, WO 03/24439, WO 03/42160, WO 03/42164, WO 03/72539, WO 03/91204, WO 03/93219, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083, WO 04/80964, EP1460064, WO 04/087142, WO 04/089892, EP 01477167, US 2004/0242622, US 2004/0229904, WO 04/108675, WO 04/108676, WO 05/033121, WO 05/040103, WO 05/044787, WO 05/058867, WO 05/065650, WO 05/066140, WO 05/07908, US 2005/5159448, US 2005/171147, WO 05/077361, WO 05/084640, WO 05/089760, WO 05/090287, WO 05/090288, WO 05/092860, WO 05/092887, US 2005/182091, US 2005/209227, US 2005/215542, US 2005/215590, EP 1574501, US05/256115, WO 05/102350 and US05/277632.

Suitable bronchodilatory drugs include anticholinergic or antimuscarinic agents, in particular

ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, US 2005/171147, US 2005/182091, WO 01/04118, WO 02/00652, WO 02/51841, WO 02/53564, WO 03/00840, WO 03/33495, WO 03/53966, WO 03/87094, WO 04/018422, WO 04/05285 and WO 05/077361.

Suitable dual anti-inflammatory and bronchodilatory drugs include dual beta-2 adrenoceptor agonist/muscarinic antagonists such as those disclosed in US 2004/0167167, US 2004/0242622, US 2005/182092, WO 04/74246 WO 04/74812, WO 04/089892 and US05/256114.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/099807 and WO 04/026841.

Other useful combinations of agents of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro —N,N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873. In accordance with the foregoing, the invention also provides a method for the treatment of a condition mediated byresponsive to activation of the adenosine A2a receptor, for example an inflammatory or allergic condition, particularly an inflammatory or obstructive airways disease, which comprises administering to a subject, particularly a human subject, in need thereof a compound of formula I in free form or in the form of a pharmaceutically acceptable salt. In another aspect the invention provides a compound of formula II, in free form or in the form of a pharmaceutically acceptable salt, for use in the manufacture of a medicament for the treatment of a condition mediated byresponsive to activation of the adenosine A2a receptor, particularly an inflammatory or obstructive airways disease.

### Formulation and Administration

The agents of the invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, for example in the treatment of atopic dermatitis; or rectally, for example in the treatment of inflammatory bowel disease.

In a further aspect, the invention also provides a pharmaceutical composition comprising a compound of formula (I) in free form or in the form of a pharmaceutically acceptable salt, optionally together with a pharmaceutically acceptable diluent or carrier therefor. The composition may contain a co-therapeutic agent such as an anti-inflammatory, broncho-dilatory, antihistamine or anti-tussive drug as hereinbefore described. Such compositions may be prepared using conventional diluents or excipients and techniques known in the galenic art. Thus oral dosage forms may include tablets and capsules.

Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula II having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it preferably contains, for example, the compound of formula (I) either dissolved, or suspended, in a vehicle containing water, a co-solvent such as ethanol or propylene glycol and a stabiliser, which may be a surfactant.

The invention includes (A) a compound of formula (I) in inhalable form, e.g. in an aerosol or other atomisable composition or in inhalable particulate, e.g. micronised, form, (B) an inhalable medicament comprising a compound of formula (I) in inhalable form; (C) a pharmaceutical product comprising a compound of formula (I) in inhalable form in association with an inhalation device; and (D) an inhalation device containing a compound of formula (I) in inhalable form.

Dosages of compounds of formula (I) employed in practising the present invention will of course vary depending, for example, on the particular condition to be treated, the effect desired and the mode of administration. In general, suitable daily dosages for administration by inhalation are of the order of 0.005 to 10 mg, while for oral administration suitable daily doses are of the order of 0.05 to 100 m.

The invention is illustrated by the following Examples of formula (I).

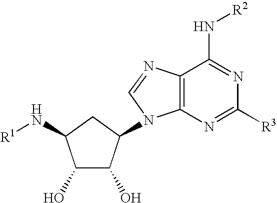

are shown in Table 1 below.

TABLE 1

| | | | | MH+ or |
|---|---|---|---|---|
| Ex. | R¹ | R² | R³ | MH+/2 |
| 1 | 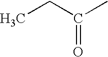 | 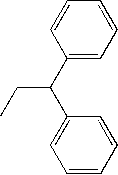 | 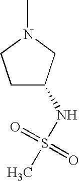 | 649.4 |
| 2 | 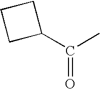 | 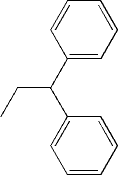 | 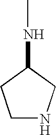 | 597.3 |
| 3 | 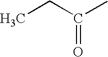 | 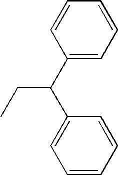 | 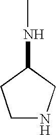 | 571.5 |
| 4 | 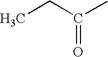 | 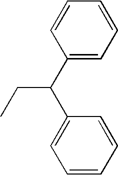 | 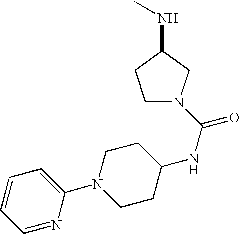 | 388.0 |
| 5 | 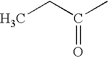 | 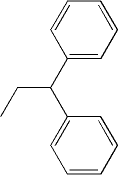 | 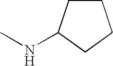 | 570.5 |
| 6 | 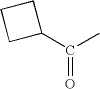 | 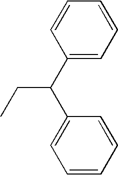 | 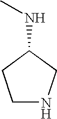 | 597.3 |
| 7 | 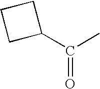 | 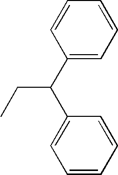 | 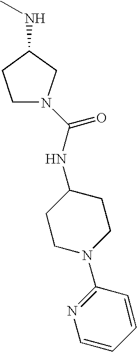 | 800.6 |
| 8 | 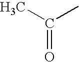 | 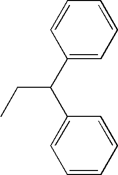 | 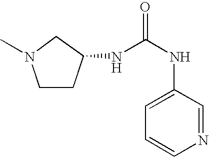 | 677.5 |
| 9 | 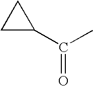 | 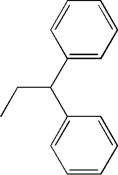 | 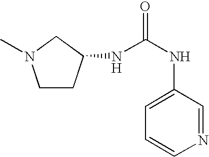 | 703.8 |
| 10 | 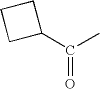 | 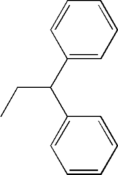 | 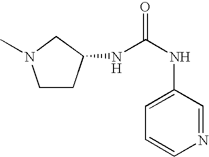 | 717.5 |
| 11 | 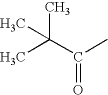 | 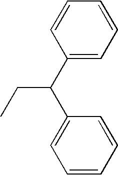 | 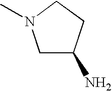 | 599.46 |
| 12 | 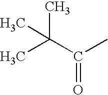 | 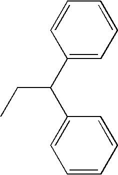 | 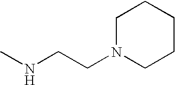 | 641.46 |
| 13 | 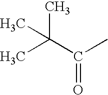 | 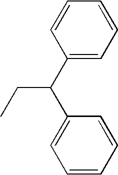 | 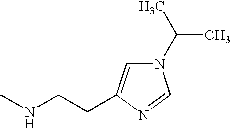 | 666.47 |
| 14 | 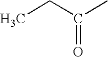 | 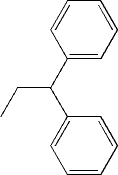 | 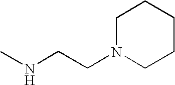 | 625.25 |
| 15 | 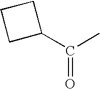 | 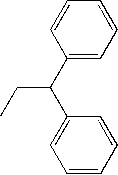 | 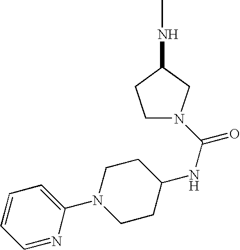 | 800.5 |

### PREPARATION OF INTERMEDIATE COMPOUNDS

Abbreviations used are as follows: CDI is 1,1′-carbonyldiimidazole, DCM is dichloromethane, DIPEA is diisopropylethylamine, DMAP is 4-dimethylaminopyridine, DMF is dimethyl-formamide, DMSO is dimethylsulfoxide, LCMS is liquid chromatographic mass spectroscopy, TEA is triethylamine, TFA is trifluoroacetic acid, THF is tetrahydrofuran, EtOH is ethanol, IPA is iso-propylalcohol and TLC is thin-layer chromatography.

### Intermediate A

### (R)-3-Amino-pyrrolidine-1-carboxylic acid (3,4,5,6-tetrahydro-2H-[1,2′]bipyridinyl-4-yl)-amide trifluoroacetate

### A1: Imidazole-1-carboxylic acid (3,4,5,6-tetrahydro-2H-[1,2′]bipyridinyl-4-yl)-amide

A suspension comprising CDI (2.29 g, 14 mmol) and triethylamine (3.8 ml, 27 mmol) in dry DCM (20 ml) is treated portionwise over 5 minutes with 3,4,5,6-tetrahydro-2H-[1,2′]bipyridinyl-4-ylamine dihydrochloride (prepared using the procedure described in international patent application WO 01/94368) (2.88 g, 13 mmol). The reaction mixture is stirred at room temperature for 4.5 hours to yield the title compound as a 0.43 M solution in DCM.

### A2: (R)-1-(3,4,5,6-Tetrahydro-2H-[1,2′]bipyridinyl-4-ylcarbamoyl)-pyrrolidin-3-yl]-carbamic acid tert-butyl ester trifluoroacetate

To a solution of (R)-Pyrrolidin-3-yl-carbamic acid tert-butyl ester (1.2 g, 6.45 mmol) in toluene/isopropanol (30 ml of 2:1 mixture) is added imidazole-1-carboxylic acid (3,4,5,6-tetrahydro-2H-[1,2′]bipyridinyl-4-yl)-amide (A1) (25 ml of a 0.43 M solution, 10.75 mmol).

The reaction mixture is stirred at room temperature for three days and then the solvent is removed in vacuo. Purification by C-18 reverse phase column chromatography eluting with acetonitrile:water:TFA (0.1%) (gradient of 0 to 100% acetonitrile) yields the title compound. MS (ES+) m/e 390.3 (MH⁺).

### A3: (R)-3-Amino-pyrrolidine-1-carboxylic acid (3,4,5,6-tetrahydro-2H-[1,2′]bipyridinyl-4-yl)-amide trifluoroacetate

A solution of (R)-1-(3,4,5,6-Tetrahydro-2H-[1,2′]bipyridinyl-4-ylcarbamoyl)-pyrrolidin-3-ylq-carbamic acid tert-butyl ester trifluoroacetate (2.13 g, 4.24 mmol) in DCM (10 ml) is treated with TFA (4 ml). The reaction mixture is stirred at room temperature over night and then the solvent was removed. The resulting crude is purified by C-18 reverse phase column chromatography eluting with acetonitrile:water:TFA (0.1%) (gradient of 0 to 100% acetonitrile) to yield the title compound.

### Intermediate C

### Imidazole-1-carboxylic acid (3,4,5,6-tetrahydro-2H-[1,2′]bipyridinyl-4-yl)-amide

A stirred solution of CDI (1.1 g, 6.77 mmol) in DCM (100 ml) is treated with 3,4,5,6-tetrahydro-2H-[1,2′]bipyridinyl-4-ylamine (WO 9965895 EP 21973) (1 g, 5.64 mmol in 50 ml of DCM) added dropwise over 30 minutes. The reaction mixture is stirred at room temperature for 15 minutes to yield the title compound as a 10 mg/ml solution in DCM. The compound is used in solution in subsequent reactions. This solution consists of the imidazole-urea intermediate (C) together with variable amounts of the corresponding isocyanate and imidazole. This solution is used in the subsequent steps since the imidazole-urea intermediate and isocyanate intermediate are equally suitable as precursors to ureas.

### Intermediate J

### N-{(1S,2R,3S,4R)-4-[2-Chloro-6-(2,2-diphenyl-ethylamino)-purin-9-yl]-2,3-dihydroxy-cyclopentyl}-propionamide

### J1: (1S,4R)-4-(2,6-Dichloro-purin-9-yl)-cyclopent-2-enol

2,6-Dichloropurine (10 g, 52.90 mmol), (1S,4R)-cis 4-acetoxy-2-cyclopenten-1-ol (10 g. 70.40 mmol), tris(dibenzylideneacetone)dipalladium(0) (3.20 g, 3.50 mmol) and polymer supported triphenylphosphine (3 mmol/g, 11.60 g, 35.00 mmol) are placed in an oven-dried flask under an atmosphere of argon. Dry deoxygenated THF (80 ml) is added and the reaction mixture is stirred gently for 5 minutes. Triethylamine (20 ml) is added and the reaction mixture is stirred at 50° C. The reaction is shown to be complete by LCMS after 1 hour. The reaction mixture is allowed to cool, filtered and the solvent is removed in vacuo. The title compound is obtained after purification by flash column chromatography (silica, dichloromethane/methanol 25:1). ¹H nmr (CDCl₃, 400 MHz); 8.30 (s, 1H), 6.40 (m, 1H), 5.90 (m, 1H), 5.50 (m, 1H), 4.95 (m, 1H), 3.05 (m, 1H), 2.10 (m, 1H), MS (ES+) m/e 271 (MH⁺).

### J2: Carbonic acid (1S,4R)-4-(2,6-dichloro-purin-9-yl)-cyclopent-2-enyl ester ethyl ester

(1S,4R)-4-(2,6-Dichloro-purin-9-yl)-cyclopent-2-enol (9.5 g, 35.05 mmol) is placed in an oven-dried flask under an atmosphere of argon. Dry THF (200 mL) is added followed by dry pyridine (5.54 g, 70.1 mmol). Ethyl chloroformate (15.21 g, 140.2 mmol) is added slowly so that the temperature does not rise above 40° C. and the reaction mixture is stirred at room temperature. The reaction is shown to be complete by LCMS after 1 hour. The solvent is removed in vacuo and the residue is partitioned between dichloromethane (200 mL) and water (200 mL). The organic layer is washed with water (150 ml) and brine (150 ml), dried over MgSO₄, filtered and the solvent is removed in vacuo. The title compound is obtained after crystallisation from methanol. ¹H nmr (CDCl₃, 400 MHz); 8.20 (s, 1H), 6.45 (m, 1H), 6.25 (m, 1H), 5.75 (m, 1H), 5.70 (m, 1H), 4.25 (q, 2H), 3.20 (m, 1H), 2.05 (m, 1H), 1.35 (t, 3H), MS (ES+) m/e 343 (MH⁺).

### J3: Di-Boc-[(1S,4R)-4-(2,6-dichloro-purin-9-yl)-cyclopent-2-enyl]-amine

Carbonic acid (1S,4R)-4-(2,6-dichloro-purin-9-yl)-cyclopent-2-enyl ester ethyl ester (2.5 g, 7.29 mmol), di-t-butyl iminodicarboxylate (1.74 g, 8.02 mmol), tris(dibenzylideneacetone)dipalladium(0) (0.33 g, 0.36 mmol) and triphenylphosphine (0.29 g, 1.09 mmol) are placed in an oven-dried flask under an atmosphere of argon. Dry deoxygenated THF (30 ml) is added and the reaction mixture is stirred at room temperature. The reaction is shown to be complete by LCMS after 3 hours. The solvent is removed in vacuo and the title compound is obtained after purification by flash column chromatography (silica, ethyl acetate/isohexane 4:1) ¹H nmr (CDCl₃, 400 MHz); 8.70 (s, 1H), 6.20 (m, 1H), 5.85 (m, 1H), 5.80 (m, 1H), 5.40 (m, 1H), 3.20 (m, 1H), 2.15 (m, 1H), 1.55 (s, 18H), MS (ES+) m/e 470 (MH⁺).

### J4: (1S,2R,3S,5R)-3-(Di-Boc-amino)-5-(2,6-dichloro-purin-9-yl)-cyclopentane-1,2-diol

The title compound is prepared from di-Boc-[(1S,4R)-4-(2,6-dichloro-purin-9-yl)-cyclopent-2-enyl]-amine using a procedure analogous to that use to prepare (1R,2S,3R,5S)-3-(6-{[bis-(4-methoxy-phenyl)-methyl]-amino}-2-chloro-purin-9-yl)-5-(di-Boc-amino)-cyclopentane-1,2-diol (Intermediate in the preparation of Intermediate ZA). ¹H nmr (CDCl₃, 400 MHz); 8.35 (s, 1H), 4.80 (m, 1H), 4.70 (m, 1H), 4.50 (m, 1H), 3.85 (m, 1H), 3.75 (m, 1H), 3.10 (m, 1H), 2.75 (m, 1H), 2.55 (m, 1H), 1.55 (s, 18H), MS (ES+) m/e 504 (MH⁺).

### J5: (1S,2R,3S,5R)-3-Amino-5-(2,6-dichloro-purin-9-yl)-cyclopentane-1,2-diol trifluoroacetate

A solution of (1S,2R,3S,5R)-3-(Di-Boc-amino)-5-(2,6-dichlorourin-9-yl)-cyclopentane-1,2-diol (0.550 g, 1.09 mmol) in DCM (4 ml) is treated with TFA (2 ml) and stirred at room temperature for 2 hours. The solvent is removed in vacuo and afford the title product which is used in the next step without further purification.

MS (ES+) m/e 304 (MH⁺).

### J6: N-[(1S,2R,3S,4R)-4-(2,6-Dichloro-purin-9-yl)-2,3-dihydroxy-cyclopentyl]-propionamide

A solution of 1S,2R,3S,5R)-3-amino-5-(2,6-dichloro-purin-9-yl)-cyclopentane-1,2-diol trifluoroacetate (0.304 g, 1.00 mmol) in THF (10 ml) is treated with DIPEA (0.387 g, 3.00 mmol) followed by propionyl chloride (0.093 g, 1.00 mmol). The reaction mixture is stirred at room temperature for 2 hours. The solvent is removed in vacuo and the title compound is obtained after purification by reverse phase column chromatography (Isolute™ C18, 0-100% acetonitrile in water-0.1% TFA). MS (ES+) m/e 360 (MH⁺).

### J7: N-{(1S,2R,3S,4R)-4-[2-Chloro-6-(2,2-diphenyl-ethylamino)-purin-9-yl]-2,3-dihydroxy-cyclopentyl}-propionamide

N-[(1S,2R,3S,4R)-4-(2,6-Dichloro-purin-9-yl)-2,3-dihydroxy-cyclopentyl]-propionamide (160 mg, 0.44 mmol) is dissolved in THF (5 ml) under an atmosphere of argon. Diisopropylamine (69 mg, 0.53 mmol) is added followed by 2,2-diphenylethylamine (96 mg, 0.49 mmol) and the reaction mixture is stirred at 50° C. The reaction is shown to be complete by LCMS after 2 hours. The solvent is removed in vacuo and the title compound is obtained after purification by reverse phase column chromatography (Isolute™ C18, 0-100% acetonitrile in water-0.1% TFA). ¹H nmr (MeOD, 400 MHz); 8.00 (s, 1H), 7.40-7.15 (m, 10H), 4.75 (m, 1H), 4.60 (m, 1H), 4.50 (m, 1H), 4.20 (m, 3H), 3.95 (m, 1H), 2.85 (m, 1H), 2.40 (q, 2H), 2.10 (m, 1H), 1.20 (t, 3H), MS (ES+) m/e 521 (MH⁺).

The final compound of Intermediate J may also be prepared using the following process:

### JJ1: {2-Chloro-9-[(1R,4S)-4-(di-Boc-amino)-cyclopent-2-enyl]-9H-purin-6-yl}-(2,2-diphenyl-ethyl)-amine

(1S,2R,3S,5R)-3-(Di-Boc-amino)-5-(2,6-dichloro-purin-9-yl)-cyclopentane-1,2-diol (13.0 g, 27.66 mmol) is dissolved in THF (250 ml) under an atmosphere of argon.

Diisopropylamine (4.28 g, 33.19 mmol) is added followed by 2,2-diphenylethylamine (6.0 g, 30.43 mmol) and the reaction mixture is stirred at 50° C. The reaction is shown to be complete by LCMS after 18 hours. The solvent is removed in vacuo and the reaction mixture is partitioned between dichloromethane (250 ml) and 0.1M HCl (250 ml). The organic layer is washed with water (200 ml) and brine (200 ml), dried over MgSO₄, filtered and the solvent is removed in vacuo to give the title compound. ¹H nmr (CDCl₃, 400 MHz); 8.05 (s, 1H), 7.30-7.10 (m, 10H), 6.00 (m, 1H), 5.70 (m, 2H), 5.60 (m, 1H), 5.20 (m, 1H), 4.30 (m, 1H), 4.20 (m, 1H), 3.65 (m, 1H), 3.05 (m, 1H), 2.00 (m, 1H), 1.70 (m, 1H), 1.40 (s, 18H), MS (ES+) m/e 631 (MH⁺).

### JJ2: (1R,2S,3R,5S)-3-[2-Chloro-6-(2,2-diphenyl-ethylamino)-purin-9-yl]-5-(di-Boc-amino)-cyclopentane-1,2-diol

A solution of {2-Chloro-9-[(1R,4S)-4-(di-Boc-amino)-cyclopent-2-enyl]-9H-purin-6-yl}-(2,2-diphenyl-ethyl)-amine (2.9 g, 4.6 mmol) in THF (60 ml) is treated with 4-methyl morpholine N-oxide (1.1 g, 9.3 mmol) and osmium tetroxide (4% solution in water) (6 ml) and the mixture is stirred at room temperature for 48 hours. The solvent is removed under reduced pressure and the residue is purified by column chromatography on silica gel eluting with a gradient system of methanol:dichloromethane (0:100 by volume) gradually changing to methanol:dichloromethane (4:96 by volume) to afford the title compound. LCMS (electrospray): m/z [MH⁺] 665.34

### JJ3: (1S,2R,3S,5R)-3-Amino-5-[2-chloro-6-(2,2-diphenyl-ethylamino)-purin-9-yl]-cyclopentane-1,2-diol trifluoroacetate

(1R,2S,3R,5S)-3-[2-Chloro-6-(2,2-diphenyl-ethylamino)-purin-9-yl]-5-(di-Boc-amino)-cyclopentane-1,2-diol (10.3 g, 15.50 mmol) is dissolved in dichloromethane (50 ml). TFA (25 ml) is added and the reaction mixture is stirred at room temperature. The reaction is shown to be complete by LCMS after 2 hours. The solvent is removed in vacuo to give the title compound. ¹H nmr (MeOD, 400 MHz); 7.90 (s, 1H), 7.30-7.10 (m, 10H), 4.65 (m, 1H), 4.50 (m, 1H), 4.40 (m, 1H), 4.20 (m, 1H), 4.10 (m, 2H), 3.50 (m, 1H), 2.75 (m, 1H), 2.15 (m, 1H), MS (ES+) m/e 465 (MH⁺).

### JJ4: N-{(1S,2R,3S,4R)-4-[2-Chloro-6-(2,2-diphenyl-ethylamino)purin-9-yl]-2,3-dihydroxy-cyclopentyl}-propionamide

(1S,2R,3S,5R)-3-Amino-5-[2-chloro-6-(2,2-diphenyl-ethylamino)-purin-9-yl]-cyclopentane-1,2-diol trifluoroacetate (9.50 g, 16.42 mmol) and diisopropylethylamine (6.36 g, 49.27 mmol) are placed in a flask with dry THF (150 ml). Propionyl chloride (1.52 g, 16.42 mmol) is added dropwise and the reaction mixture is stirred at room temperature. The reaction is shown to be complete by LCMS after 1 hour. The solvent is removed in vacuo and the residue is partitioned between dichloromethane (250 ml) and water (250 ml). The organic layer is washed with water (200 ml) and brine (200 ml), dried over MgSO₄, filtered and the solvent is removed in vacuo. The solid is recrystallised from 1,2-dichloroethane to give the title compound. ¹H nmr (MeOD, 400 MHz); 8.00 (s, 1H), 7.40-7.15 (m, 10H), 4.75 (m, 1H), 4.60 (m, 1H), 4.50 (m, 1H), 4.20 (m, 3H), 3.95 (m, 1H), 2.85 (m, 1H), 2.40 (q, 2H), 2.10 (m, 1H), 1.20 (t, 3H), MS (ES+) m/e 521 (MH⁺).

### Intermediate K

### Cyclobutanecarboxylic acid {(1S,2R,3S,4R)-4-[2-chloro-6-(2,2-diphenyl-ethylamino)-purin-9-yl]-2,3-dihydroxy-cyclopentyl}-amide

A solution of (1S,2R,3S,5R)-3-amino-5-[2-chloro-6-(2,2-diphenyl-ethylamino)-purin-9-yl]-cyclopentane-1,2-diol hydrochloride (100 mg, 0.2 mmol) in dry THF (1 ml) is treated with diisopropylethylamine (0.17 ml, 1 mmol) and cyclobutanecarboxylic acid chloride (0.023 ml, 0.2 mmol) and the mixture is stirred at room temperature for 48 hours. The solvent is removed under reduced pressure. The residue is purified by reverse-phase chromatography eluting with a gradient system of acetonitrile (0.1% TFA): water (0.1% TFA) (0:100 by volume) gradually changing to acetonitrile (0.1% TFA): water (0.1% TFA) (100:0 by volume) to afford the title compound (51 mg). LCMS (electrospray): m/z [MH⁺] 547.26. ¹H nmr (MeOD, 400 MHz); 8.00 (s, 1H), 7.40-7.25 (m, 8H), 7.20-7.15 (m, 2H), 4.70 (m, 1H), 4.50 (m, 2H), 4.20 (m, 2H), 3.95 (m, 1H), 2.85 (m, 1H), 2.30 (m, 2H), 2.20 (m, 2H), 2.05 (m, 2H), 1.90 (m, 1H)

### Intermediate L

### {(1S,2R,3S,4R)-4-[2-((R)-3-Amino-pyrrolidin-1-yl)-6-(2,2-diphenyl-ethylamino)-purin-9-yl]-2,3-dihydroxy-cyclopentyl}-carbamic acid benzyl ester

### L1: Preparation of intermediate L1

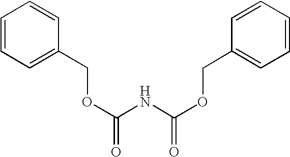

A cooled (0° C.) solution of benzyl carbamate (4.0 g, 27 mmol) in THF (100 ml) under an inert atmosphere of Argon is treated with potassium iodide (3.2 g of a 35% w/w dispersion in oil, 28 mmol) portionwise over 10 minutes. The reaction mixture is allowed to warm to room temperature over 30 minutes after which time benzyl chloroformate (5.0 g, 29 mmol) is added. After stirring at room temperature for 2 hours, the reaction is quenched with water (20 ml). The THF is removed in vacuo and the resulting mixture is partioned between EtOAc and 2M HCl. The organic portion is separated and washed with brine, dried (MgSO₄) and concentrated in vacuo. The resulting oil is purified by chromatography on silica eluting with 1:3 EtOAc/iso-hexane to yield a product which is recrystallised from DCM/iso-hexane to afford the title product.

### L2: Preparation of intermediate L2

A solution comprising carbonic acid (1S,4R)-4-(2,6-dichloro-purin-9-yl)-cyclopent-2-enyl ester ethyl ester (J2) (2.0 g, 5.83 mmol), Intermediate L1 (2.2 g, 7.58 mmol) and triphenyl phosphine (229 mg, 0.9 mmol) in THF (20 ml) is stirred at room temperature for minutes. Tris(dibenzylideneacetone)dipalladium (0) (238 mg, 0.3 mmol) is added and the resulting mixture is stirred at room temperature for 1.5 hours. The solvent is removed in vacuo and the crude product is purified by chromatography on silica eluting with MeOH/DCM (gradient of 0 to 1% MeOH) to yield the title compound.

### L3: Preparation of intermediate L3

This compound is prepared analogously to 2-chloro-9-[(1R,4S)-4-(di-Boc-amino)-cyclopent-2-enyl]-9H-purin-6-yl}-(2,2-diphenyl-ethyl)-amine (JJ1) by replacing (1S,2R,3S,5R)-3-(Di-Boc-amino)-5-(2,6-dichloro-purin-9-yl)-cyclopentane-1,2-diol (Intermediate J4) with Intermediate L2.

### L4: Preparation of intermediate L4

This compound is prepared analogously to (1R,2S,3R,5S)-3-[2-chloro-6-(2,2-diphenyl-ethylamino)-purin-9-yl]-5-(di-Boc-amino)-cyclopentane-1,2-diol (JJ2) by replacing {2-Chloro-9-[(1R,4S)-4-(di-Boc-amino)-cyclopent-2-enyl]-9H-purin-6-yl}-(2,2-diphenyl-ethyl)-amine with Intermediate L3.

### L5: {(R)-1-[9-((1R,2S,3R,4S)-4-Benzyloxycarbonylamino-2,3-dihydroxy-cyclopentyl)-6-(2,2-diphenyl-ethylamino)-9H-purin-2-yl]-pyrrolidin-3-yl}-carbamic acid tert-butyl ester

A suspension of Intermediate L4 (1.03 g, 1.4 mmol) and (3R)-(+)-3-(Boc-amino)pyrrolidine (1.03 g, 5.5 mmol) in acetonitrile (2 ml) is treated with sodium iodide (ca. 2 mg) and then heated using microwave radiation in a Personal Chemistry Emrys™ Optimizer microwave reactor at 160° C. After 1 hour, the solvent is removed in vacuo and the crude residue is partioned between DCM and 0.2 M HCl. The organic layer is separated and the aqueous portion is extracted with DCM. The combined organic extracts are washed with saturated sodium bicarbonate solution, water, brine, dried (MgSO₄) and concentrated in vacuo to afford the title compound as a brown oil. MS (ES+) m/e 745 (MH⁺).

### L6: {(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-carbamic acid benzyl ester

A solution of {(R)-1-[9-((1R,2S,3R,4S)-4-benzyloxycarbonylamino-2,3-dihydroxy-cyclopentyl)-6-(2,2-diphenyl-ethylamino)-9H-purin-2-yl]-pyrrolidin-3-yl}-carbamic acid tert-butyl ester (Intermediate L5) (1.24 g, 1.7 mmol) in MeOH (3 ml) is treated with 4M HCl in dioxane (5 ml) and stirred at room temperature for 2 hours. The solvent is removed in vacuo and purification is carried out by reverse phase column chromatography (Isolute™ C18, 0-100% acetonitrile in water-0.1% HCl). The fractions are collected and the MeCN is removed in vacuo. The remaining aqueous portion is basified with saturated sodium bicarbonate solution and extracted with DCM. The combined organic extracted are dried (MgSO₄) and concentrated in vacuo to afford the title product. MS (ES+) m/e 649 (MH⁺).

### Intermediate V

### 2-(1-Isopropyl-1H-imidazol-4-yl)-ethylamine

This compound is prepared from 2-isopropyl-5-oxo-5,6,7,8-tetrahydro-imidazo[1,5-c]pyrimidin-2-ium iodide by the procedure of Rahul Jain and Louis A. Cohen Tetrahedron 1996, 52, 5363. ¹H nmr (MeOD, 400 MHz); 7.60 (s, 1H), 6.95 (s, 1H), 4.40 (m, 1H), 2.90 (t, 2H), 2.70 (t, 2H), 1.45 (d, 6H).

### PREPARATION OF EXAMPLES

### Example 1

N-{(1S,2R,3S,4R)-4-[6-(2,2-Diphenyl-ethylamino)-2-((R)-3-methanesulfonylamino-pyrrolidin-1-yl)-purin-9-yl]-2,3-dihydroxy-cyclopentyl}-propionamide

### Step 1: {(R)-1-[9-((1R,2S,3R,4S)-2,3-Dihydroxy-4-propionylamino-cyclopentyl)-6-(2,2-diphenyl-ethylamino)-9H-purin-2-yl]-pyrrolidin-3-yl}-carbamic acid tert-butyl ester trifluoroacetate

A reaction mixture comprising N-{(1S,2R,3S,4R)-4-[2-chloro-6-(2,2-diphenyl-ethylamino)-purin-9-yl]-2,3-dihydroxy-cyclopentyl}-propionamide (Intermediate J) (2.5 g, 4.80 mmol) and (3R)-(+)-(3-Boc-amino)pyrrolidine (2.5 g, 13.6 mmol) in DMSO (8 ml) is heated at 100° C. overnight. The resulting mixture is purified by reverse phase column chromatography (Isolute™ C18, 0-100% MeOH in water-0.1% TFA) to yield the title product.

### Step 2: N-{(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-propionamide

{(R)-1-[9-((1R,2S,3R,4S)-2,3-Dihydroxy-4-propionylamino-cyclopentyl)-6-(2,2-diphenyl-ethylamino)-9H-purin-2-yl]-pyrrolidin-3-yl}-carbamic acid tert-butyl ester trifluoroacetate (3.22 g, 4.80 mmol) is dissolved in 1.25 M HCl in MeOH (60 ml, 75 mmol) and left to stir at room temperature overnight. The solvent is removed in vacuo and the crude product is dissolved in a minimal volume of EtOH/saturated sodium carbonate solution and purified by reverse phase column chromatography (Isolute™ C18, 0-100% MeOH in water) to yield the title product.

### Step 3: N-{(1S,2R,3S,4R)-4-[6-(2,2-Diphenyl-ethylamino)-2-((R)-3-methanesulfonylamino-pyrrolidin-1-yl)-purin-9-yl]-2,3-dihydroxy-cyclopentyl}-propionamide

A solution comprising N-{(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-propionamide (0.03 g, 0.04 mmol) in DCM (1 ml) is treated with TEA (0.012 ml, 0.088 mmol)) followed by methane sulphonyl chloride (0.03 ml, 0.04 mmol). After the reaction mixture is allowed to stand at room temperature overnight and the solvent is removed in vacuo. Purification by reverse phase column chromatography (Isolute™ C18, 0-100% acetonitrile in water—0.1% TFA) yields the title product.

### Example 2

### Cyclobutanecarboxylic acid {(1S,2R,3S,4R)-4-[6-(2,2-diphenyl-ethylamino)-2-((R)-pyrrolidin-3-ylamino)-purin-9-yl]-2,3-dihydroxy-cyclopentyl}-amide trifluoroacetate

### Step 1: Cyclobutanecarboxylic acid {(1S,2R,3S,4R)-4-[2-((R)-1-benzyl-pyrrolidin-3-ylamino)-6-(2,2-diphenyl-ethylamino)-purin-9-yl]-2,3-dihydroxy-cyclopentyl}-amide trifluoroacetate

A solution comprising cyclobutanecarboxylic acid {(1S,2R,3S,4R)-4-[2-chloro-6-(2,2-diphenyl-ethylamino)-purin-9-yl]-2,3-dihydroxy-cyclopentyl}-amide (Intermediate K) (80 mg, 0.15 mmol) in NMP/MeCN (0.5 ml of a 1:1 mixture) is treated with (R)-1-benzyl-3-aminopyrrolidine (129 mg, 0.73 mmol) followed by sodium iodide (22 mg, 0.15 mmol).

The reaction mixture is heated using microwave radiation in a Personal Chemistry Emrys™ Optimizer microwave reactor at 200° C. for 135 minutes. The solvent is removed in vacuo and purification by C-18 reverse phase column chromatography eluting with acetonitrile:water:TFA (0.1%) (gradient of 0 to 100% acetonitrile) yields the title compound.

### Step 2: Cyclobutanecarboxylic acid {(1S,2R,3S,4R)-4-[6-(2,2-diphenyl-ethylamino)-2-((R)-pyrrolidin-3-ylamino)-purin-9-yl]-2,3-dihydroxy-cyclopentyl}-amide trifluoroacetate

A solution of cyclobutanecarboxylic acid {(1S,2R,3S,4R)-4-[2-((R)-1-benzyl-pyrrolidin-3-ylamino)-6-(2,2-diphenyl-ethylamino)-purin-9-yl]-2,3-dihydroxy-cyclopentyl}-amide trifluoroacetate (52 mg, 0.06 mmol) in EtOH (2 ml) is treated with palladium hydroxide (20% on carbon) (47 mg, 90 mol %) followed by ammonium formate (20 mg, 0.32 mmol) and heated at reflux for 4 hours. The solvent is removed in vacuo and purification by C-18 reverse phase column chromatography eluting with acetonitrile:water:TFA (0.1%) (gradient of 0 to 100% acetonitrile) yields the title compound.

### Example 3

### N-{(1S,2R,3S,4R)-4-[6-(2,2-Diphenyl-ethylamino)-2-((R)-pyrrolidin-3-ylamino)-purin-9-yl]-2,3-dihydroxy-cyclopentyl}-propionamide trifluoroacetate

The title compound is prepared analogously to Example 2 by replacing cyclobutanecarboxylic acid {(1S,2R,3S,4R)-4-[2-chloro-6-(2,2-diphenyl-ethylamino)-purin-9-yl]-2,3-dihydroxy-cyclopentyl}-amide (Intermediate K) with N-{(1S,2R,3S,4R)-4-[2-chloro-6-(2,2-diphenyl-ethylamino)purin-9-yl]-2,3-dihydroxy-cyclopentyl}-propionamide (Intermediate J).

### Example 4

### (R)-3-[9-((1R,2S,3R,4S)-2,3-Dihydroxy-4-propionylamino-cyclopentyl)-6-(2,2-diphenyl-ethylamino)-9H-purin-2-ylamino]-pyrrolidine-1-carboxylic acid (3,4,5,6-tetrahydro-2H-[1,2′]bipyridinyl-4-yl)-amide trifluoroacetate

To a solution comprising N-{(1S,2R,3S,4R)-4-[6-(2,2-diphenyl-ethylamino)-2-((R)-pyrrolidin-3-ylamino)-purin-9-yl]-2,3-dihydroxy-cyclopentyl}-propionamide trifluoroacetate (Example 3) (0.693 g, 1.01 mmol) in iso-propanol (5 ml) is added TEA (0.282 ml, 2.02 mmol) followed by imidazole-1-carboxylic acid (3,4,5,6-tetrahydro-2H-[1,2′]bipyridinyl-4-yl)-amide (Intermediate C) (30 ml of a 10 mg/ml solution in DCM, 1.11 mmol). After the reaction mixture has stirred at room temperature overnight, the solvent is removed in vacuo and purification of the crude by reverse phase column chromatography (Isolute™ C18, 0-100% acetonitrile in water-0.1% TFA) yields the title product.

### Example 5

### N-{(1S,2R,3S,4R)-4-[2-cyclopentylamino-6-(2,2-diphenylethlamino)-purin-9-yl]-2,3-dihydroxy-cyclopentyl}-propionamide trifluoroacetate

This compound is prepared analogously to cyclobutanecarboxylic acid {(1S,2R,3S,4R)-4-[2-((R)-1-benzyl-pyrrolidin-3-ylamino)-6-(2,2-diphenyl-ethylamino)-purin-9-yl]-2,3-dihydroxy-cyclopentyl}-amide trifluoroacetate (Example 2 step 1) by replacing cyclobutanecarboxylic acid {(1S,2R,3S,4R)-4-[2-chloro-6-(2,2-diphenyl-ethylamino)-purin-9-yl]-2,3-dihydroxy-cyclopentyl}-amide (Intermediate K) with N-{(1S,2R,3S,4R)-4-[2-chloro-6-(2,2-diphenyl-ethylamino)-purin-9-yl]-2,3-dihydroxy-cyclopentyl}-propionamide (Intermediate J) and by replacing (R)-1-benzyl-3-aminopyrrolidine with cyclopentylamine.

### Example 6

Cyclobutanecarboxylic acid {(1S,2R,3S,4R)-4-[6-(2,2-diphenyl-ethylamino)-2-((S)-pyrrolidin-3-ylamino)-purin-9-yl]-2,3-dihydroxy-cyclopentyl}-amide hydrochloride:

### Step 1: (S)-3-[9-[(1R,2S,3R,4S)-4-(Cyclobutanecarbonyl-amino)-2,3-dihydroxy-cyclopentyl]-6-(2,2-diphenyl-ethylamino)-9H-purin-2-ylamino]-pyrrolidine-1-carboxylic acid tert-butyl ester trifluoroacetate

A reaction mixture comprising cyclobutanecarboxylic acid {(1S,2R,3S,4R)-4-[2-chloro-6-(2,2-diphenyl-ethylamino)-purin-9-yl]-2,3-dihydroxy-cyclopentyl}-amide (Intermediate K) (120 mg, 0.22 mmol), (S)-3-amino-1N-Boc-pyrrolidine (408 mg, 2.2 mmol), sodium iodide (33 mg, 0.22 mmol) and NMP/MeCN (0.5 ml of a 1:1 mixture) is heated using microwave radiation in a Personal Chemistry Emrys™ Optimizer microwave reactor at 160° C. for 195 minutes. Purification by C-18 reverse phase column chromatography eluting with acetonitrile:water:TFA (0.1%) (gradient of 0 to 100% acetonitrile) yields the title compound.

### Step 2: Cyclobutanecarboxylic acid {(1S,2R,3S,4R)-4-[6-(2,2-diphenyl-ethylamino)-2-((S)-pyrrolidin-3-ylamino)-purin-9-yl]-2,3-dihydroxy-cyclopentyl}-amide hydrochloride

(S)-3-[9-[(1R,2S,3R,4S)-4-(Cyclobutanecarbonyl-amino)-2,3-dihydroxy-cyclopentyl]-6-(2,2-diphenyl-ethylamino)-9H-purin-2-ylamino]pyrrolidine-1-carboxylic acid tert-butyl ester trifluoroacetate (55 mg, 0.07 mmol) is dissolved in 1.25 M HCl in MeOH (1 ml) an allowed to stand at room temperature for 2 days. The solvent is removed in vacuo to afford the title compound.

### Example 7

### (S)-3-[9-[(1R,2S,3R,4S)-4-(Cyclobutanecarbonyl-amino)-2,3-dihydroxy-cyclopentyl]-6-(2,2-diphenyl-ethylamino)-9H-purin-2-ylamino]-pyrrolidine-1-carboxylic acid (3,4,5,6-tetrahydro-2H-[1,2′]bipyridinyl-4-yl)-amide trifluoroacetate

Cyclobutanecarboxylic acid {(1S,2R,3S,4R)-4-[6-(2,2-diphenyl-ethylamino)-2-((S)-pyrrolidin-3-ylamino)-purin-9-yl]-2,3-dihydroxy-cyclopentyl}-amide hydrochloride (Example 27) (20 mg, 0.03 mmol) and TEA (9 μl, 0.06 mmol) in IPA (0.5 ml) is treated with imidazole-1-carboxylic acid (3,4,5,6-tetrahydro-2H-[1,2′]bipyridinyl-4-yl)-amide (Intermediate C) (1.41 ml of a 10 mg/ml solution in DCM) and stirred at room temperature overnight. The solvent is removed in vacuo and purification by reverse phase column chromatography (Isolute™ C18, 0-100% acetonitrile in water-0.1% TFA) affords the title compound.

### Example 8

### N-((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-acetamide hydrochloride

### Step 1: (1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-carbamic acid benzyl ester trifluoroacetate

A solution comprising {(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-carbamic acid benzyl ester (Intermediate L) (0.1 g, 0.15 mmol), pyridine-3-isocyanate (0.02 g, 0.17 mmol) and TEA (0.017 g, 0.17 mmol) in THF (2 ml) is stirred at room temperature overnight. The solvent is removed in vacuo and purification is carried out by reverse phase column chromatography (Isolute™ C18, 0-100% acetonitrile in water-0.1% TFA). The fractions are collected and the MeCN is removed in vacuo. The remaining aqueous portion is basified with saturated sodium bicarbonate solution and extracted with DCM. The combined organic extracted are dried (MgSO₄) and concentrated in vacuo to afford the title product. MS (ES+) m/e 769 (MH⁺).

### Step 2: 1-{(R)-1-[9-((1R,2S,3R,4S)-4-Amino-2,3-dihydroxy-cyclopentyl)-6-(2,2-diphenyl-ethylamino)-9H-purin-2-yl]-pyrrolidin-3-yl}-3-pyridin-3-yl-urea

To a solution of ((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-carbamic acid benzyl ester trifluoroacetate (35 mg, 46 μmol) in ethanol (1 ml) under an inert atmosphere of Argon is added 10% palladium on carbon (10 mg). The reaction mixture is purged with Argon and placed under a positive atmosphere of hydrogen overnight after which time, the mixture is filtered through celite and the catalyst washed with ethanol. The organic portions are combined and concentrated in vacuo to yield the title compound. MS (ES+) m/e 635 (MH⁺).

### Step 3: N-((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-acetamide hydrochloride

A solution of 1-{(R)-1-[9-((1R,2S,3R,4S)-4-amino-2,3-dihydroxy-cyclopentyl)-6-(2,2-diphenyl-ethylamino)-9H-purin-2-yl]-pyrrolidin-3-yl}-3-pyridin-3-yl-urea (11 mg, 17 μmol) and DIPEA (7 mg, 54 mmol) in THF (0.5 ml) and NMP (0.1 ml) is treated with acetyl chloride (1.5 mg, 19 μmol) in THF (0.15 ml). After stirring at room temperature for 30 minutes, the solvent is removed in vacuo and the crude product is dissolved in MeOH. Saturated sodium bicarbonate solution is added and the reaction mixture is left overnight. The reaction mixture is purified by reverse phase column chromatography (Isolute™ C18, 0-100% acetonitrile in water—0.1% HCl) to afford the title product. MS (ES+) m/e 678 (MH⁺)

### Example 9 and 10

These compounds namely,

Cyclopropanecarboxylic acid ((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-amide (Example 9) and
Cyclobutanecarboxylic acid ((1S,2R,3S,4R)-4-{6-(2,2-diphenyl-ethylamino)-2-[(R)-3-(3-pyridin-3-yl-ureido)-pyrrolidin-1-yl]-purin-9-y}-2,3-dihydroxy-cyclopentyl)-amide (Example 10)
are prepared analogously to N-((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-acetamide hydrochloride (Example 8) by replacing acetyl chloride with the appropriate acid chloride.

### Example 11

### N-{(1S,2R,3S,4R)-4-[2-((R)-3-Amino-pyrrolidin-1-yl)-6-(2,2-diphenyl-ethylamino)-purin-9-yl]-2,3-dihydroxy-cyclopentyl}-2,2-dimethyl-propionamide

### Step 1: N-{(1S,2R,3S,4R)-4-[2-Chloro-6-(2,2-diphenyl-ethylamino)-purin-9-yl]-2,3-dihydroxy-cyclopentyl}-2,2-dimethyl-propionamide

This compound is prepared analogously to N-{(1S,2R,3S,4R)-4-[2-chloro-6-(2,2-diphenyl-ethylamino)-purin-9-yl]-2,3-dihydroxy-cyclopentyl}-propionamide (JJ4) by replacing propionyl chloride with trimethylacetyl chloride.

### Step 2: N-{(1S,2R,3S,4R)-4-[2-((R)-3-Amino-pyrrolidin-1-yl)-6-(2,2-diphenyl-ethylamino)-purin-9-yl]-2,3-dihydroxy-cyclopentyl}-2,2-dimethyl-propionamide

A solution of N-{(1S,2R,3S,4R)-4-[2-chloro-6-2,2-diphenyl-ethylamino)-purin-9-yl]-2,3-dihydroxy-cyclopentyl}-2,2-dimethyl-propionamide (20 mg, 0.04 mmol) and in NMP/MeCN (0.5 ml of a 1:1 mixture) is treated with sodium iodide (6 mg, 0.04 mmol) and (R)-pyrrolidin-3-ylamine (34 mg, 0.4 mmol). The reaction mixture is heated using microwave radiation in a Personal Chemistry Emrys™Optimizer microwave reactor at 200° C. for 30 minutes. The solvent is removed in vacuo and purification by C-18 reverse phase column chromatography eluting with acetonitrile:water:TFA (0.1%) (gradient of 0 to 100% acetonitrile) yields the title compound.

### Examples 12-14

These compounds namely,

N-{(1S,2R,3S,4R)-4-[6-(2,2-Diphenyl-ethylamino)-2-(2-piperidin-1-yl-ethylamino)-purin-9-yl]-2,3-dihydroxy-cyclopentyl}-2,2-dimethyl-propionamide trifluoroacetate (Example 12), N-((1S,2R,3S,4R)-4-{6-(2,2-Diphenyl-ethylamino)-2-[2-(1-isopropyl-1H-imidazol-4-yl)-ethylamino]-purin-9-yl}-2,3-dihydroxy-cyclopentyl)-2,2-dimethyl-propionamide (Example 13) and
Cyclopropanecarboxylic acid {(1S,2R,3S,4R)-4-[6-(2,2-diphenyl-ethylamino)-2-(2-piperidin-1-yl-ethylamino)-purin-9-yl]-2,3-dihydroxy-cyclopentyl}-amide (Example 14) are prepared analogously to Example 11 by replacing (R)-pyrrolidin-3-ylamine with the appropriate amine.

### Example 15

### (R)-3-[9-[(1R,2S,3R,4S)-4-(cyclobutanecarbonyl-amino)-2,3-dihydroxy-cyclopentyl]-6-(2,2-diphenyl-ethylamino)-9H-purin-2-ylamino]-pyrrolidine-1-carboxylic acid (3,4,5,6-tetrahydro-2H-[1,2′]bipyridinyl-4-yl)-amide trifluoroacetate

This compound is prepared analogously to cyclobutanecarboxylic acid {(1S,2R,3S,4R)-4-[2-((R)-1-benzyl-pyrrolidin-3-ylamino)-6-(2,2-diphenyl-ethylamino)-purin-9-yl]-2,3-dihydroxy-cyclopentyl}-amide trifluoroacetate (Example 2 step 1) by replacing (R)-1-benzyl-3-aminopyrrolidine with (R)-3-amino-pyrrolidine-1-carboxylic acid (3,4,5,6-tetrahydro-2H-[1,2′]bipyridinyl-4-yl)-amide (Intermediate A).

1. A compound of formula (I) 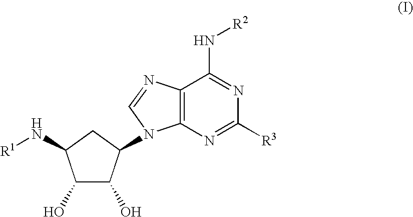
in free or salt form,
wherein R¹, R² and R³ are

| R¹ | R² | R³ |
|---|---|---|
| 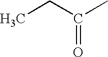 | 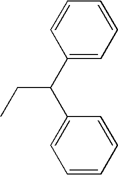 | 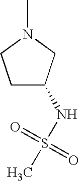 |
| 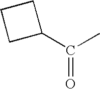 | 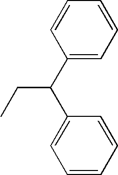 | 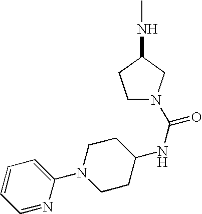 |
| 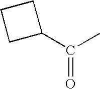 | 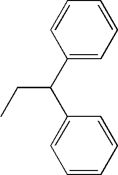 | 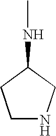 |
| 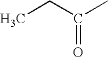 | 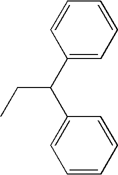 | 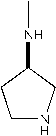 |
| 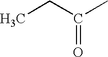 | 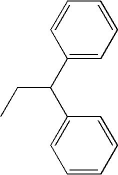 | 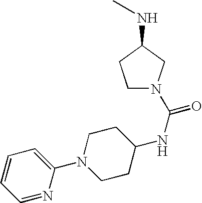 |
| 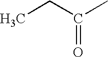 | 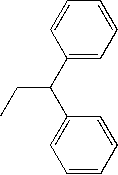 | 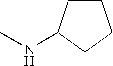 |
| 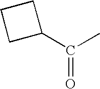 | 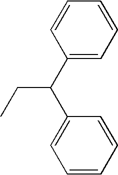 | 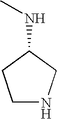 |
| 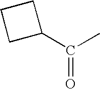 | 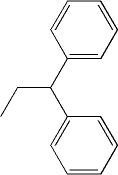 | 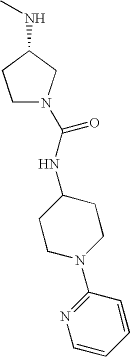 |
| 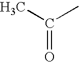 | 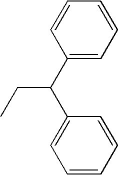 | 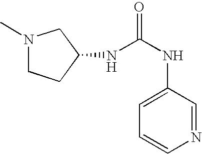 |
| 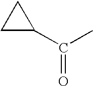 | 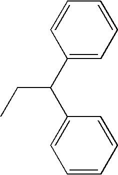 | 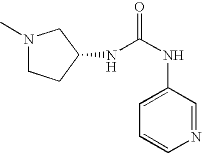 |
| 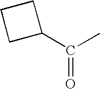 | 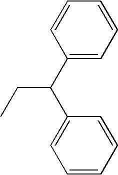 | 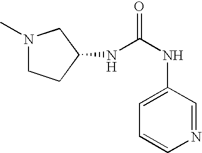 |
| 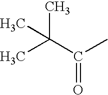 | 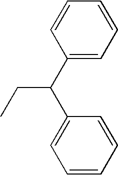 | 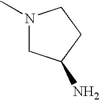 |
| 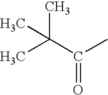 | 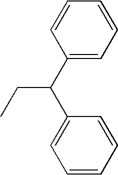 | 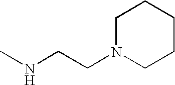 |
| 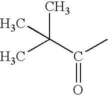 | 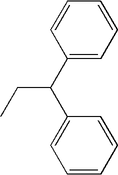 | 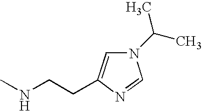 |
| 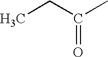 | 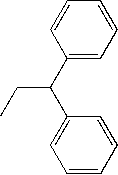 | 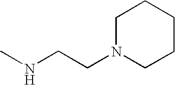 |

2. A compound according to claim 1 for use as a pharmaceutical.

3. A compound according to claim 1 in combination with an anti-inflammatory, bronchodilatory, antihistamine or anti-tussive drug substance, said compound and said drug substance being in the same or different pharmaceutical composition.

4. A pharmaceutical composition comprising as active ingredient a compound according to claim 1, optionally together with a pharmaceutically acceptable diluent or carrier.

5. A pharmaceutical composition according to claim 4, further comprising an anti-inflammatory, bronchodilatory, antihistamine or anti-tussive drug substance.

6. Use of a compound according to claim 1 for the manufacture of a medicament for the treatment of a condition mediated by activation of the adenosine A2a receptor.

7. Use of a compound according to claim 1 for the manufacture of a medicament for the treatment of an inflammatory or obstructive airways disease.

8. A method of preparing a compound of formula (I) as defined in claim 1 in free or salt form which comprises:
(i) reacting a compound of formula (II) 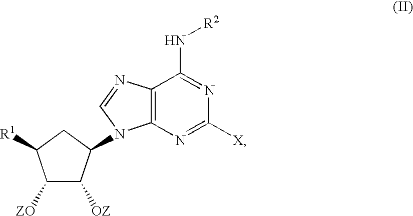
wherein
R¹, and R² are as defined in claim 1;
Z is H or a protecting group; and
X is a leaving group,
with a compound of formula (III)
H—R³ (III),
wherein
R³ is as defined in claim 1; and
(ii) removing any protecting groups and recovering the resultant compound of formula (I), in free or pharmaceutically acceptable salt form.
